(19) United States
(12) Patent Application Publication
(10) Pub. No.: US 2022/0336108 A1
(43) Pub. Date: Oct. 20, 2022

(54) Enhanced Disease Projections with Mobility

(71) Applicant: International Business Machines Corporation, Armonk, NY (US)

(72) Inventors: George Sirbu, Saline, MI (US); Ujwal Reddy Moramganti, Ashburn, VA (US); Sayali Navalekar, Westford, MA (US); Vishrawas Gopalakrishnan, Cambridge, MA (US); Ajay Ashok Deshpande, Pleasantville, NY (US); Sarah Kefayati, San Francisco, CA (US); Pan Ding, New York, NY (US); Raman Srinivasan, Plano, TX (US); Xuan Liu, Yorktown Heights, NY (US); James H. Kaufman, San Jose, CA (US)

(21) Appl. No.: 17/230,751

(22) Filed: Apr. 14, 2021

(51) Int. Cl. G16H 50/80; G06N 20/20; G06N 5/04; G16H 50/20; G16H 50/70

(52) U.S. Cl. CPC G16H 50/80; G06N 20/20; G06N 5/04; G16H 50/20; G16H 50/70; G06N 5/003

(57) ABSTRACT

A mechanism is provided in a data processing system to implement a model pipeline for predicting changes in disease transmission rate using a spatial temporal epidemiological model. The mechanism receives input data comprising disease case data for a disease and mobility data and prepares the input data to generate a training dataset, a validation dataset, and a test dataset. A feature selection module performs feature selection on the input data to select a first set of features for a binary classification computer model, a second set of features for a three-level classification computer model, and a third set of features for a regression computer model. The mechanism determines a future predicted transmission rate value for the subsequent time period using the binary classification computer model, the three-level classification computer model, and the regression computer model and generates disease projections for the subsequent time period based on the future predicted transmission rate value and the spatial temporal epidemiological model.

### BACKGROUND

The present application relates generally to an improved data processing apparatus and method and more specifically to mechanisms for predicting changes in disease transmission rates based on mobility using artificial intelligence and machine learning.

The classic models of the spread of infectious disease are compartment models, so-called because they involve the use of compartments of individuals organized by infective status. The SIR (susceptible, infectious, recovered) and SEIR (susceptible, exposed, infectious, recovered) models were used to simulate the spread of SARS (severe acute respiratory syndrome). The models are named according to the compartments used:

Susceptible (S) people have no immunity from the disease;

Infectious (I) people have the disease and can spread it to others;

Exposed (E) people have contracted the disease but are not yet infection spreaders; and,

Recovered (or removed or resistant) (R) people have recovered from the disease and are immune to further infection.

These models are reasonably predictive for infectious diseases that are transmitted from human to human and where recovery may confer lasting resistance, like in cases of measles, mumps, and rubella, or resistance until a new variant or loss of immunity against the pathogen occurs, like in the case of flu. The variables (S, I, R) represent the number of people in each compartment at a particular time. To represent that the number of susceptible, infectious, and recovered individuals may vary over time, even if the total population size remains constant, the model makes the precise numbers a function of time: S(t), I(t), and R(t). For a specific disease in a specific population, these functions may be worked out to predict possible outbreaks and bring them under control.

The equations governing the changes in the respective compartments S(t) and I(t) are functions of a transmission rate (beta). A problem associated with modeling the spread of infectious disease is estimating the times at which the transmission rate changes and estimating the transmission rate values. Sudden increase or decrease are referred to as inflection points. Increases in the transmission rate are referred to as elbows. Decreases in the transmission rate are referred to as knees. Current models leverage case data to detect likely changes in transmission rate parameters, determine the model parameters, and generate case projections.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system for predicting changes in disease transmission rate. The method comprises receiving input data comprising disease case data for a disease and mobility data. The method further comprises preparing, by an auto-tuning training module, a spatial temporal epidemiological model and associated parameters, including a transmission rate parameter and preparing, by a data preparation module executing within the model pipeline, the input data to generate a training dataset, a validation dataset, and a test dataset. The method further comprises performing, by a feature selection module executing within the model pipeline, feature selection on the input data to select a first set of features for a binary classification computer model, a second set of features for a three-level classification computer model, and a third set of features for a regression computer model. The method further comprises training, by a training module executing within the model pipeline, the binary classification computer model for predicting whether a transmission rate of the disease will increase or remain unchanged in a subsequent time period using the mobility data based on the first set of features; training, by the training module, the three-level classification computer model for predicting whether the transmission rate will decrease, increase, or remain unchanged in the subsequent time period using the mobility data based on the second set of features; and training, by the training module, the regression computer model for predicting a transmission rate value in the subsequent time period using the mobility data based on the third set of features. The method further comprises determining a future predicted transmission rate value for the subsequent time period using the binary classification computer model, the three-level classification computer model, and the regression computer model. The method further comprises generating disease projections for the subsequent time period based on the future predicted transmission rate value and the spatial temporal epidemiological model.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a pictorial representation of an example distributed data processing system in which aspects of the illustrative embodiments may be implemented;

FIG. 2 is a block diagram of just one example data processing system in which aspects of the illustrative embodiments may be implemented;

FIG. 3 is a block diagram depicting an approach for generating case projections based on case data in accordance with an illustrative embodiment;

FIG. 4 is a block diagram depicting a mechanism for generating case projections based on case data with mobility in accordance with an illustrative embodiment;

FIG. 5 is a block diagram illustrating a model pipeline for computerized prediction of changes in disease transmission rate based on mobility data in accordance with an illustrative embodiment;

FIG. 6 illustrates features and sources of features for building models to predict changes in transmission rate based on mobility in accordance with an illustrative embodiment;

FIG. 7 is a flowchart illustrating operation of a model pipeline for computerized prediction of changes in disease transmission rate based on mobility data in accordance with an illustrative embodiment;

FIG. 8 is a flowchart illustrating operation of a mechanism for hyper-parameter tuning for training models for beta predictions with mobility in accordance with an illustrative embodiment; and

FIG. 9 is a flowchart illustrating operation of a mechanism for predicting transmission rate value based on mobility in accordance with an illustrative embodiment.

### DETAILED DESCRIPTION

Disease case projection computer models use only case data. Sometimes, there is not enough signal in the tail end of recent case data to pick up underlying disease dynamics. This may result in case projection models missing an upcoming second wave or overestimating or underestimating an ongoing second wave.

The illustrative embodiments use other signals, such as mobility and other population characteristics, to improve modeling of tail end dynamics, which is captured by the transmission rate parameter (beta) in the spatial temporal epidemiological model. Specifically, the illustrative embodiments use mobility and other data to build a model to predict beta changes at the tail end. The illustrative embodiments build a binary classification computer model to predict if beta increases or not in a subsequent time period (e.g., 14 days) and builds a three-level classification model to predict if the beta increases, decreases, or remains unchanged in the subsequent time period. The illustrative embodiments also build a regression model to predict a new beta value in the subsequent time period. The illustrative embodiments then change the beta parameter in the spatial temporal epidemiological model in the subsequent time interval according to the prediction from the classification models and the regression model. The illustrative embodiments then evaluate performance of the computerized models. Thus, the illustrative embodiments improve computerized predictive models by combining multiple models that use mobility and other population characteristics to more accurately predict changes in transmission rate and use that to subsequently predict new cases using the spatial temporal epidemiological model.

Before beginning the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a”, “at least one of”, and “one or more of” regarding particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein regarding describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

Moreover, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

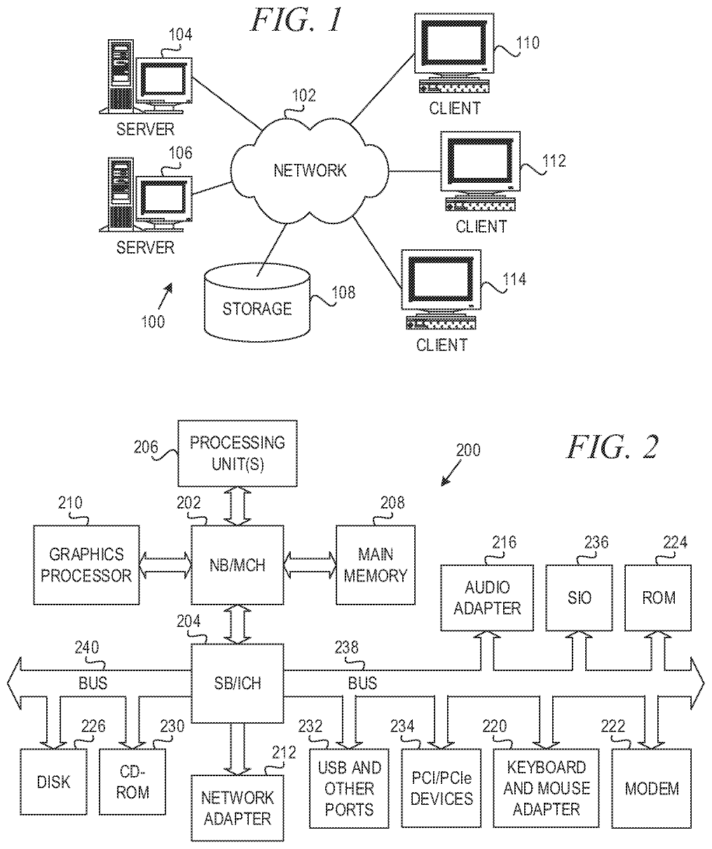

The illustrative embodiments may be utilized in many different types of data processing environments. In order to provide a context for the description of the specific elements and functionality of the illustrative embodiments, FIGS. 1 and 2 are provided hereafter as example environments in which aspects of the illustrative embodiments may be implemented. It should be appreciated that FIGS. 1 and 2 are only examples and are not intended to assert or imply any limitation with regard to the environments in which aspects or embodiments of the present invention may be implemented. Many modifications to the depicted environments may be made without departing from the spirit and scope of the present invention.

FIG. 1 depicts a pictorial representation of an example distributed data processing system in which aspects of the illustrative embodiments may be implemented. Distributed data processing system 100 may include a network of computers in which aspects of the illustrative embodiments may be implemented. The distributed data processing system 100 contains at least one network 102, which is the medium used to provide communication links between various devices and computers connected together within distributed data processing system 100. The network 102 may include connections, such as wire, wireless communication links, or fiber optic cables.

In the depicted example, server 104 and server 106 are connected to network 102 along with storage unit 108. In addition, clients 110, 112, and 114 are also connected to network 102. These clients 110, 112, and 114 may be, for example, personal computers, network computers, or the like. In the depicted example, server 104 provides data, such as boot files, operating system images, and applications to the clients 110, 112, and 114. Clients 110, 112, and 114 are clients to server 104 in the depicted example. Distributed data processing system 100 may include additional servers, clients, and other devices not shown.

In the depicted example, distributed data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational, and other computer systems that route data and messages. Of course, the distributed data processing system 100 may also be implemented to include several different types of networks, such as for example, an intranet, a local area network (LAN), a wide area network (WAN), or the like. As stated above, FIG. 1 is intended as an example, not as an architectural limitation for different embodiments of the present invention, and therefore, the elements shown in FIG. 1 should not be considered limiting regarding the environments in which the illustrative embodiments of the present invention may be implemented.

As shown in FIG. 1, one or more of the computing devices, e.g., server 104, may be specifically configured to implement a computerized epidemiological model for predicting changes in transmission rate based on mobility data using artificial intelligence and machine learning. The configuring of the computing device may comprise the providing of application specific hardware, firmware, or the like to facilitate the performance of the operations and generation of the outputs described herein regarding the illustrative embodiments. The configuring of the computing device may also, or alternatively, comprise the providing of software applications stored in one or more storage devices and loaded into memory of a computing device, such as server 104, for causing one or more hardware processors of the computing device to execute the software applications that configure the processors to perform the operations and generate the outputs described herein regarding the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general-purpose computing device. Moreover, as described hereafter, the implementation of the mechanisms of the illustrative embodiments improves the functionality of the computing device and provides a useful and concrete result that facilitates beta prediction with mobility using computerized spatial temporal epidemiological models.

As noted above, the mechanisms of the illustrative embodiments utilize specifically configured computing devices, or data processing systems, to perform the operations for disease transmission projections with mobility. These computing devices, or data processing systems, may comprise various hardware elements which are specifically configured, either through hardware configuration, software configuration, or a combination of hardware and software configuration, to implement one or more of the systems/subsystems described herein. FIG. 2 is a block diagram of just one example data processing system in which aspects of the illustrative embodiments may be implemented. Data processing system 200 is an example of a computer, such as server 104 in FIG. 1, in which computer usable code or instructions implementing the processes and aspects of the illustrative embodiments of the present invention may be located and/or executed to achieve the operation, output, and external effects of the illustrative embodiments as described herein.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 may be connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 may be connected to SB/ICH 204.

An operating system runs on processing unit 206. The operating system coordinates and provides control of various components within the data processing system 200 in FIG. 2. As a client, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 200.

As a server, data processing system 200 may be, for example, an IBM eServer™ System p® computer system, Power™ processor-based computer system, or the like, running the Advanced Interactive Executive (AIX™) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and may be loaded into main memory 208 for execution by processing unit 206. The processes for illustrative embodiments of the present invention may be performed by processing unit 206 using computer usable program code, which may be located in a memory such as, for example, main memory 208, ROM 224, or in one or more peripheral devices 226 and 230, for example.

A bus system, such as bus 238 or bus 240 as shown in FIG. 2, may be comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, may include one or more devices used to transmit and receive data. A memory may be, for example, main memory 208, ROM 224, or a cache such as found in NB/MCH 202 in FIG. 2.

As mentioned above, in some illustrative embodiments the mechanisms of the illustrative embodiments may be implemented as application specific hardware, firmware, or the like, application software stored in a storage device, such as HDD 226 and loaded into memory, such as main memory 208, for executed by one or more hardware processors, such as processing unit 206, or the like. As such, the computing device shown in FIG. 2 becomes specifically configured to implement the mechanisms of the illustrative embodiments and specifically configured to perform the operations and generate the outputs described hereafter regarding the computer models for disease transmission projections using mobility data.

Those of ordinary skill in the art will appreciate that the hardware in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

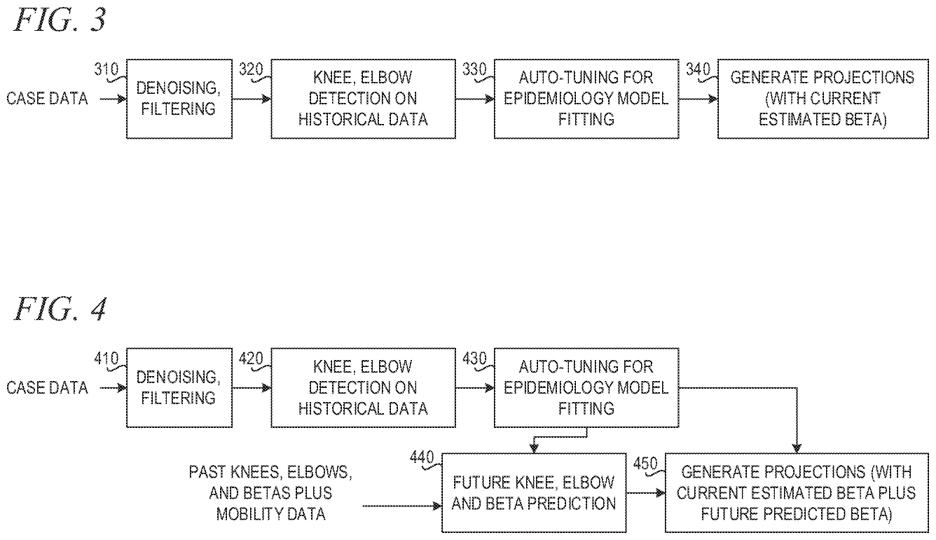

FIG. 3 is a block diagram depicting a mechanism for generating case projections based on case data in accordance with an illustrative embodiment. The mechanism receives disease case data, such as case data for coronavirus disease 2019 (COVID-19). The mechanism performs denoising and filtering (block 310) and then performs knee/elbow detection on historical data to identify inflection points (block 320). This component of the mechanism detects knees/elbows (decreases and increases, respectively) to account for likely changes in transmission rate parameters (betas).

The mechanism further performs auto-tuning for spatial temporal epidemiological model fitting (block 330). This component of the mechanism determines model parameters including beta values. Then, the mechanism generates case projections with a current estimated transmission rate (beta) (block 340). This approach detects beta changes in hindsight, e.g., five days after a change.

FIG. 4 is a block diagram depicting a mechanism for generating case projections based on case data with mobility in accordance with an illustrative embodiment. The mechanism leverages mobility data plus additional data, such as population density and demographics, to build a predictive computer model to predict upcoming changes in transmission rate.

The mechanism receives disease case data, such as case data for coronavirus disease 2019 (COVID-19). The mechanism performs denoising and filtering (block 410) and then performs knee/elbow detection on historical data to identify inflection points (block 420). This component of the mechanism detects knees/elbows (decreases and increases, respectively) to account for likely changes in transmission rate parameters (betas) in the existing case data.

The mechanism further performs auto-tuning for spatial temporal epidemiological model fitting (block 430). This component of the mechanism determines model parameters including beta values.

The mechanism also receives mobility data plus parameters estimates from epidemiological model from block 430. The mechanism then predicts future knees, elbows, and betas (block 440). This component uses historical knowledge about mobility plus other data and past changes in beta to build a predictive model for future beta value. The mechanism then generates case projections with current estimated beta plus future predicted beta (block 450).

The mechanism of the illustrative embodiment may use various classification or regression approaches to predict increases (elbows) and decreases (knees) in transmission rate (beta) and to predict transmission rate values. This approach uses mobility to predict beta changes ahead of time.

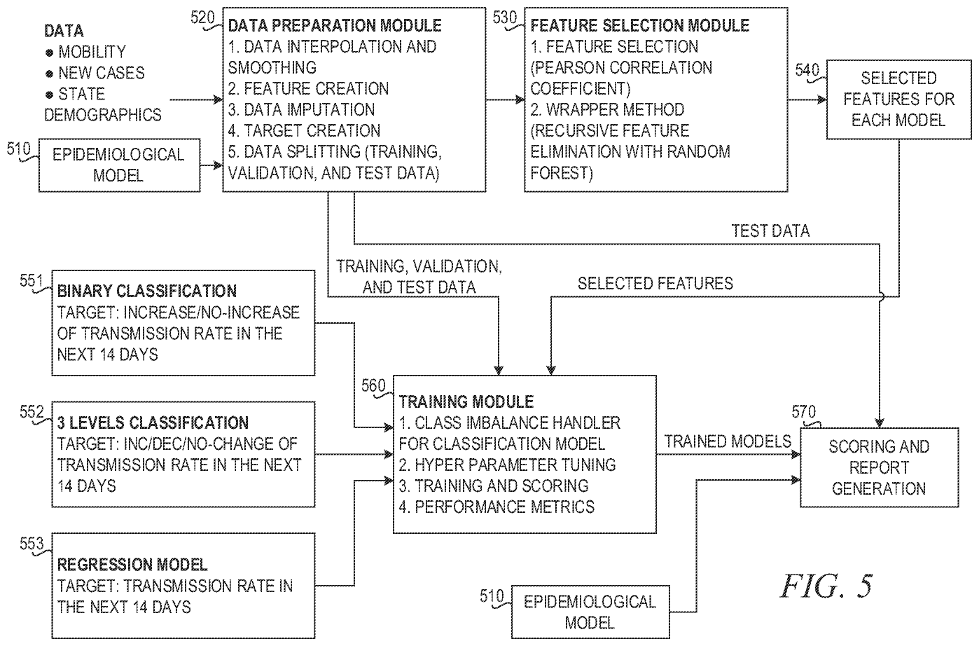

FIG. 5 is a block diagram illustrating a model pipeline for computerized prediction of changes in disease transmission rate based on mobility data in accordance with an illustrative embodiment. The model pipeline comprises an iterative process including mobility data analysis and impact, correlation analysis, spatial temporal epidemiological model data generation, data preparation, feature generation, classification modeling, regression modeling, and integration into epidemiological model for scoring.

The model pipeline receives data including mobility data, new cases, and state demographics. The model pipeline also receives parameters estimates from epidemiological model 510, which may be the model shown in FIG. 3 and described above. The epidemiological model 510 is also referred to herein as an estimated beta model. Data preparation module 520 performs data interpolation and smoothing, feature creation, data imputation, target creation, and data splitting (training data, validation data, and test data). Data preparation module 520 provides training, validation, and test data to training module 560 and sends test data to report generation module 570.

In an example embodiment, feature selection module 530 performs feature selection using Pearson correlation coefficient and wrapper method with recursive feature elimination (RFE). Feature selection module 530 generates selected features 540 for each model, which are provided to training module 560. The Pearson correlation coefficient is a measure of linear correlation between two sets of data. In one example embodiment, the wrapper method may use recursive feature elimination with random forest. Random forests are an ensemble learning method for classification, regression, and other tasks that operate by constructing a multitude of decision trees at training time and outputting the class that is the mode of the classes (classification) or mean/average prediction (regression) of the individual trees. First, the estimator is trained on the initial set of features and the importance of each feature is obtained. Then, the least important features are pruned from the current set of features. This procedure is recursively repeated on the pruned set until the desired number of features to select is eventually reached. The feature selection process is based on a specific machine learning algorithm to fit on a given dataset. It follows a greedy search approach by evaluating all the possible combinations of features against the evaluation criterion.

The model pipeline trains a binary classification model 551, three-level classification model 552, and regression model 553. In an example embodiment, binary classification model 551 has a target of increase/no-increase of transmission rate in the next fourteen days. In one example embodiment, three-level classification model 552 has a target of increase, decrease, or no-change of transmission rate in the next fourteen days. In an example embodiment, regression model 553 has a target of a transmission rate value in the next fourteen days.

Training module 560 trains models 551-553. Training module 560 includes a class imbalance handler for classification model, hyper parameter tuning, training and scoring, and evaluation of performance metrics. The class distributions may be imbalanced for both binary and three-level classification problems. In order to handle class imbalance, the algorithm may offer a weighting mechanism to weight each class. Training module 560 provides the trained models to report generator module 570, which performs scoring and report generation based on results of the trained models 551-553 and results generated by data preparation module 520.

The training process for training the models 551-553 comprises defining each model, defining a grid search for all hyper parameters, defining evaluative criteria to judge the model, training the model on the training dataset for each combination of hyper parameters in the grid search, selecting a parameter set that gives the best performance on the validation dataset, using observations from all the days up to the end of the period corresponding to the validation dataset and performing re-training using the selected parameters, and checking model performance on the test data.

In accordance with the illustrative embodiment, the models 551-553 predict prospectively the changes in the beta parameter, which in turn will have an impact on the projection of new cases. For example, binary classification model 551 may predict whether there will be a change in beta in the next 14 days; three-level classification model 552 may predict whether the beta will increase, decrease, or stay the same in the next 14 days; and regression model 553 may estimate a new value for the beta parameter in the next 14 days.

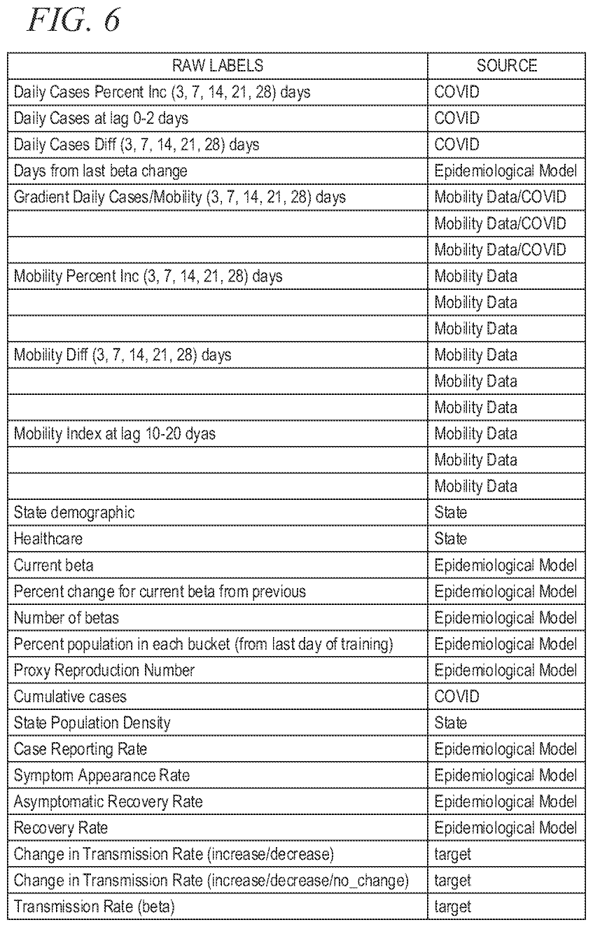

In one embodiment, the features used in the models include daily cases from the immediate past, weekly growth in daily cases from the immediate past, mobility indexes and changes in mobility in the past month, and other state demographics and healthcare access (e.g., beds per person). FIG. 6 illustrates features and sources of features for building models to predict changes in transmission rate based on mobility in accordance with an illustrative embodiment. More specifically, FIG. 6 illustrates features used to predict changes in transmission rate of COVID-19 in accordance with the illustrative embodiment. The sources for the features include COVID-19 case data from various government agencies, spatial temporal epidemiological model parameters, and mobile device mobility data. The features include the following:

Features at the state level:
state policies, population size and age distributions
access to healthcare (beds per person)

Features based on daily cases:
counts
percent increase from various lagging days
difference from various lagging days

Features based on mobility:
indexes
percent increase from various lagging days
difference from various lagging days
gradient of mobility by daily cases from various lagging days

The features also include features based on the spatial temporal epidemiological model parameters. All features based on daily cases and mobility are first transformed through smoothed weekly moving averages. Each state, each set of parameters from epidemiological model, and daily COVID cases creates one observation for training the mobility model. Features related to spatial temporal epidemiological model parameters are based on the values at the end of the corresponding training period.

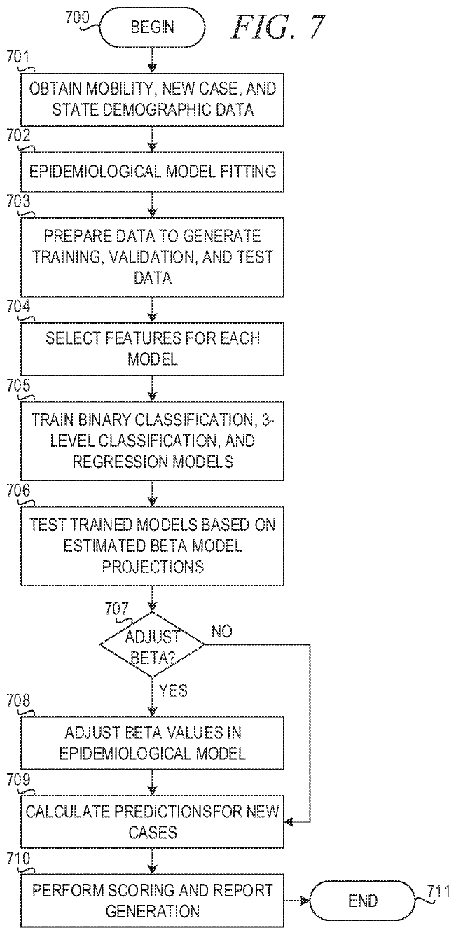

FIG. 7 is a flowchart illustrating operation of a model pipeline for computerized prediction of changes in disease transmission rate based on mobility data in accordance with an illustrative embodiment. Operation begins (block 700), and the model pipeline obtains mobility, new case, and state demographic data (block 701). The model pipeline also performs epidemiological model fitting (block 702). The model pipeline then prepares the data to generate training, validation, and test data (block 703).

The model pipeline selects features for each model (block 704) and trains a binary classification model, a three-level classification model, and a regression model using the selected features and the training, validation, and test data (block 705). The model pipeline then tests the trained models based on the estimated beta model projections (block 706).

The model pipeline then determines whether to adjust beta values based on the binary classification model, the three-level classification model, or the regression model (block 707). That is, the model pipeline evaluates criteria to judge the models, selects a parameter set that gives the best performance on the validation dataset, and re-trains the models using the selected parameters. If the model pipeline determines to adjust the models, then the model pipeline adjusts the beta values in the spatial temporal epidemiological models accordingly (block 809).

Thereafter, or if the model pipeline determines not to adjust the models, the model pipeline then calculates the predictions for new cases in the next time period based on the spatial temporal epidemiological model and the readjusted transmission rate parameter beta (block 709) and performs scoring and report generation (block 710). Thereafter, operation ends (block 711).

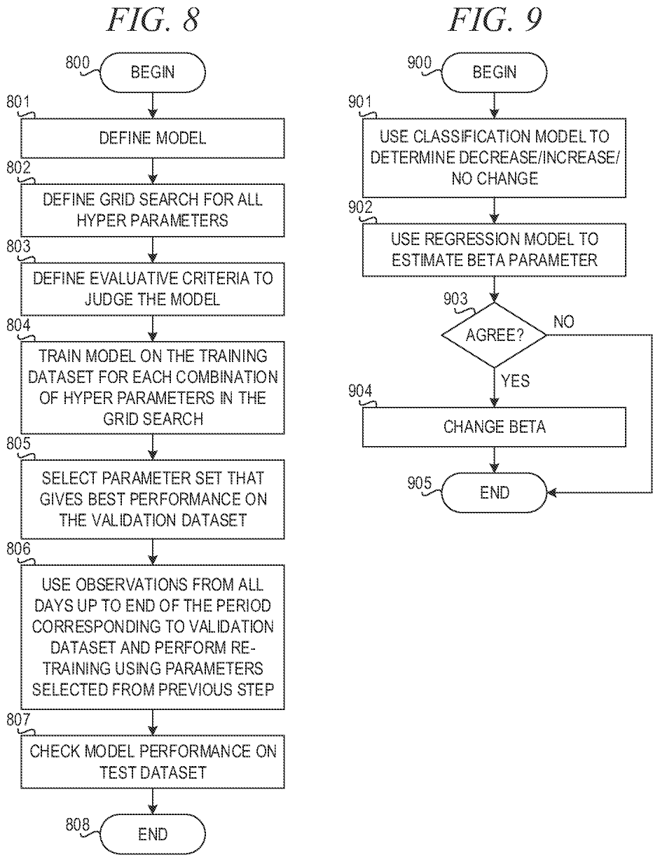

FIG. 8 is a flowchart illustrating operation of a mechanism for hyper-parameter tuning for training models for beta predictions with mobility in accordance with an illustrative embodiment. Operation begins (block 800), and the mechanism defines the model (block 801). The mechanism defines a grid search for all hyper parameters (block 802) and evaluative criteria to judge the model (block 803). The mechanism trains the model on the training dataset for each combination of hyper parameters in the grid search (block 804). The mechanism then selects the parameter set that gives the best performance on the validation dataset (block 805).

The mechanism uses observations from all days up to the end of the period corresponding to the validation dataset and performs re-training using the parameters selected from the previous step (block 806). Then, the mechanism checks the model performance on the test dataset (block 807). Thereafter, operation ends (block 808).

FIG. 9 is a flowchart illustrating operation of a mechanism for predicting transmission rate value based on mobility in accordance with an illustrative embodiment. Operation begins (block 900), and the mechanism uses the classification model to determine whether the transmission rate will decrease, increase, or remain unchanged within a subsequent time period (block 901). The mechanism uses the regression model to estimate the beta parameter during the subsequent time period (block 902).

The mechanism determines whether the classification model and the regression model agree (block 903). For example, the classification model may determine that the transmission rate increases, while the regression model may predict a transmission rate parameter that is significantly greater than the previous transmission rate value, in which case, the classification model and the regression model agree. If the models agree in block 903, then the mechanism changes the transmission rate (beta) parameter in the case projection model (block 904), and operation ends (block 905). If the models do not agree in block 903, then operation ends (block 905).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, the at least one memory comprising instructions executed by the at least one processor to cause the at least one processor to implement a model pipeline for predicting changes in disease transmission rate, the method comprising:
receiving input data comprising disease case data for a disease and mobility data;
preparing, by an auto-tuning training module, a spatial temporal epidemiological model and associated parameters, including a transmission rate parameter;
preparing, by a data preparation module executing within the model pipeline, the input data to generate a training dataset, a validation dataset, and a test dataset;
performing, by a feature selection module executing within the model pipeline, feature selection on the input data to select a first set of features for a binary classification computer model, a second set of features for a three-level classification computer model, and a third set of features for a regression computer model;
training, by a training module executing within the model pipeline, the binary classification computer model for predicting whether a transmission rate of the disease will increase or remain unchanged in a subsequent time period using the mobility data based on the first set of features;
training, by the training module, the three-level classification computer model for predicting whether the transmission rate will decrease, increase, or remain unchanged in the subsequent time period using the mobility data based on the second set of features;
training, by the training module, the regression computer model for predicting a transmission rate value in the subsequent time period using the mobility data based on the third set of features;
determining a future predicted transmission rate value for the subsequent time period using the binary classification computer model, the three-level classification computer model, and the regression computer model; and
generating disease projections for the subsequent time period based on the future predicted transmission rate value and the spatial temporal epidemiological model.

2. The method of claim 1, wherein preparing the input data comprises performing data interpolation and smoothing, performing feature creation, performing data imputation, and performing target creation.

3. The method of claim 1, wherein performing feature selection on the input data comprises performing recursive feature elimination with random forest.

4. The method of claim 1, wherein the training module comprises a class imbalance handler for handling class imbalances between the binary classification computer model and the three-level classification computer model.

5. The method of claim 1, wherein the training module performs hyper parameter tuning, training, and scoring.

6. The method of claim 1, wherein training a given model within the binary classification computer model, the three-level classification computer model, and the registration model comprises:
defining a grid search for all hyper parameters;
defining evaluation criteria to judge the given model;
training the given model on the training dataset for each combination of hyper parameters in the grid search; and
selecting a parameter set that gives a best performance of the given model on the validation dataset.

7. The method of claim 6, wherein training the given model further comprises using observations from all days up to the end of the time period corresponding to the validation dataset and performing re-training the given model using the selected parameter set.

8. The method of claim 7, wherein training the given model further comprises checking performance of the given model on the test dataset.

9. The method of claim 1, wherein determining the future predicted transmission rate value comprises:
using the binary classification computer model to determine whether the transmission rate increases, decreases, or remains unchanged;
using the regression computer model to determine the future predicted transmission rate value; and
responsive to determining the binary classification computer model and the regression computer model agree, changing a transmission rate value in a spatial temporal epidemiological model to the future predicted transmission rate value.

10. The method of claim 1, wherein the input data comprises state demographic data.

11. A computer program product comprising a computer readable storage medium having a computer readable program stored therein, wherein the computer readable program, when executed on a computing device, causes the computing device to implement a model pipeline for predicting changes in disease transmission rate, wherein the computer readable program causes the computing device to:
receive input data comprising disease case data for a disease and mobility data;
prepare, by an auto-tuning training module, a spatial temporal epidemiological model and associated parameters, including a transmission rate parameter;
prepare, by a data preparation module executing within the model pipeline, the input data to generate a training dataset, a validation dataset, and a test dataset;
perform, by a feature selection module executing within the model pipeline, feature selection on the input data to select a first set of features for a binary classification computer model, a second set of features for a three-level classification computer model, and a third set of features for a regression computer model;
train, by a training module executing within the model pipeline, the binary classification computer model for predicting whether a transmission rate of the disease will increase or remain unchanged in a subsequent time period using the mobility data based on the first set of features;
train, by the training module, the three-level classification computer model for predicting whether the transmission rate will decrease, increase, or remain unchanged in the subsequent time period using the mobility data based on the second set of features;
train, by the training module, the regression computer model for predicting a transmission rate value in the subsequent time period using the mobility data based on the third set of features;
determine a future predicted transmission rate value for the subsequent time period using the binary classification computer model, the three-level classification computer model, and the regression computer model; and
generate disease projections for the subsequent time period based on the future predicted transmission rate value and the spatial temporal epidemiological model.

12. The computer program product of claim 11, wherein preparing the input data comprises performing data interpolation and smoothing, performing feature creation, performing data imputation, and performing target creation.

13. The computer program product of claim 11, wherein performing feature selection on the input data comprises performing recursive feature elimination with random forest.

14. The computer program product of claim 11, wherein the training module comprises a class imbalance handler for handling class imbalances between the binary classification computer model and the three-level classification computer model.

15. The computer program product of claim 11, wherein the training module performs hyper parameter tuning, training, and scoring.

16. The computer program product of claim 11, wherein training a given model within the binary classification computer model, the three-level classification computer model, and the registration model comprises:
defining a grid search for all hyper parameters;
defining evaluation criteria to judge the given model;
training the given model on the training dataset for each combination of hyper parameters in the grid search; and
selecting a parameter set that gives a best performance of the given model on the validation dataset.

17. The computer program product of claim 16, wherein training the given model further comprises using observations from all days up to the end of the time period corresponding to the validation dataset and performing re-training the given model using the selected parameter set.

18. The computer program product of claim 17, wherein training the given model further comprises checking performance of the given model on the test dataset.

19. The computer program product of claim 11, wherein determining the future predicted transmission rate value comprises:
using the binary classification computer model to determine whether the transmission rate increases, decreases, or remains unchanged;
using the regression computer model to determine the future predicted transmission rate value; and
responsive to determining the binary classification computer model and the regression computer model agree, changing a transmission rate value in a spatial temporal epidemiological model to the future predicted transmission rate value.

20. An apparatus comprising:
a processor; and
a memory coupled to the processor, wherein the memory comprises instructions which, when executed by the processor, cause the processor to implement a model pipeline for predicting changes in disease transmission rate, wherein the instructions cause the processor to:
receive input data comprising disease case data for a disease and mobility data;
prepare, by an auto-tuning training module, a spatial temporal epidemiological model and associated parameters, including a transmission rate parameter;
prepare, by a data preparation module executing within the model pipeline, the input data to generate a training dataset, a validation dataset, and a test dataset;
perform, by a feature selection module executing within the model pipeline, feature selection on the input data to select a first set of features for a binary classification computer model, a second set of features for a three-level classification computer model, and a third set of features for a regression computer model;
train, by a training module executing within the model pipeline, the binary classification computer model for predicting whether a transmission rate of the disease will increase or remain unchanged in a subsequent time period using the mobility data based on the first set of features;
train, by the training module, the three-level classification computer model for predicting whether the transmission rate will decrease, increase, or remain unchanged in the subsequent time period using the mobility data based on the second set of features;
train, by the training module, the regression computer model for predicting a transmission rate value in the subsequent time period using the mobility data based on the third set of features;
determine a future predicted transmission rate value for the subsequent time period using the binary classification computer model, the three-level classification computer model, and the regression computer model; and
generate disease projections for the subsequent time period based on the future predicted transmission rate value and the spatial temporal epidemiological model.
